(19) United States
(12) Patent Application Publication
(10) Pub. No.: US 2007/0038295 A1
(43) Pub. Date: Feb. 15, 2007

(54) Artificial valve prosthesis having a ring frame

(75) Inventors: Brian C. Case, Lake Villa, IL (US); Jacob A. Flagle, Indianapolis, IN (US); Brian L. Bates, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/502,730

(22) Filed: Aug. 11, 2006

Related U.S. Application Data
(60) Provisional application No. 60/708,041, filed on Aug. 12, 2005.

(51) Int. Cl. A61F 2/24

(52) U.S. Cl. 623/2.18; 623/1.24

(57) ABSTRACT

A radially expandable artificial valve prosthesis for regulating fluid flow through a body vessel is provided. The prosthesis includes a radially expandable ring frame, at least one valve leaflet attached to the ring frame forming a valve pocket and a support structure attached to the ring frame and adapted to position the ring frame within the bodily passage. The height of the valve pocket is less than the maximum cross sectional dimension of the lumen defined by the expanded ring frame. The valve leaflet is allows fluid flow in a first, antegrade, direction and restricts flow in a second, retrograde direction.

### RELATED APPLICATIONS

This non-provisional patent application claims priority to U.S. Provisional Patent Application No. 60/708,041, filed Aug. 12, 2005, the contents of which are incorporated by reference in their entirety.

### TECHNICAL FIELD

This invention relates to medical devices, more particularly to valve prostheses and the like.

### BACKGROUND

Many vessels in animals transport fluids from one bodily location to another. In some vessels, such as mammalian veins, natural valves are positioned along the length of the vessel to permit fluid flow in a substantially unidirectional manner along the length of the vessel. These natural valves are particularly important in the lower extremities to prevent blood from pooling in the lower legs and feet during situations, such as standing or sitting, when the weight of the column of blood in the vein can act to prevent positive blood flow toward the heart. A condition, commonly known as “chronic venous insufficiency”, is primarily found in individuals where gradual dilation of the veins, thrombotic events, or other conditions prevent the leaflets of the native valves from closing properly. This leads to significant leakage of retrograde flow such that the valve is considered “incompetent”. Chronic venous insufficiency is a potentially serious condition in which the symptoms can progress from painful edema and unsightly spider or varicose veins to skin ulcerations. Elevation of the feet and compression stocking can relieve symptoms, but do not treat the underlying disease. Untreated, the disease can impact the ability of individuals to maintain their normal lifestyle.

To treat venous valve insufficiency, a number of surgical procedures have been employed to improve or replace the native valve, including placement of artificial valve prostheses. These efforts have met with limited success and have not been widely adopted as a method of treating chronic venous insufficiency. More recently, efforts have been directed towards finding a suitable self-expanding or radially-expandable artificial valve that can be placed using minimally invasive techniques, rather than requiring open surgery and its obvious disadvantages. Thus far, use of prosthetic venous valves has remained experimental only.

One common problem evident from early experiences with prosthetic valves is the formation of thrombus around the base of the leaflets, probably due at least in part to blood pooling in that region. In a natural valve, the leaflets are typically located within a sinus or enlargement in the vein. There is some evidence that the wide pockets formed between the leaflets and the walls of the sinus create vortices of flowing blood that help flush the pocket and prevent blood from stagnating and causing thrombosis around the valve leaflets, which can interfere with the function of the valve. It is thought that the stagnating blood prevents oxygen from reaching the endothelium covering the valve leaflets, leading to hypoxia of the tissues which may explain increased thrombus formation typical in that location. Expandable-frame valve prostheses typically are of a generally cylindrical in shape and lack an artificial sinus or pocket space that is sufficient for simulating these natural blood flow patterns. This is especially true when the valve leaflets of such devices are positioned at a shallow angle relative to the wall of the vessel resulting in a narrow valve pocket between the leaflet and the vessel.

Thus, prosthetic valves that mimic the sinuses naturally found surrounding native valves are desirable.

### SUMMARY

The present invention provides a valve prosthesis, such as an artificial venous valve, having a valve structure and a self-expanding or otherwise expandable support structure that upon deployment within a body lumen, such as a vein, helps create a pocket surrounding the valve leaflet of sufficient size and shape to stimulate flow patterns or vortices which facilitate clearing of the blood or other bodily fluid that would otherwise pool therein. Thus, the present invention has one or more of the following advantages: more turbulent flow, increased velocity of flow, larger and/or more numerous vortices, other factors, or a combination of the above that prevent stagnant, hypoxic areas from occurring around the valve leaflets. Furthermore, the modified flow created by the device of the present invention may also contribute to helping close the leaflets to form a seal and prevent leakage of fluid back through the valve.

In one embodiment, the present invention provides a radially expandable artificial valve prosthesis for regulating fluid flow through a body vessel. The prosthesis includes a radially expandable ring frame, at least one valve leaflet having a portion of its perimeter attached to the ring frame to form a valve pocket and a support structure attached to the ring frame and adapted to position the ring frame within the bodily vessel. The valve pocket height is less than the ring frame width. The leaflet allows fluid flow in a first, antegrade, direction and restricts flow in a second, retrograde, direction.

In one embodiment, retrograde flow positions the valve leaflet to create retrograde flow vortices sufficient to reduce stagnation of fluid in a pocket of the valve leaflet when the valve prosthesis is positioned to restrict fluid flow in the retrograde direction.

In one embodiment, the valve leaflet is attached to the ring frame by a method such as suturing, tissue welding and adhesive bonding. In another embodiment the ring frame includes a stainless steel, nickel, silver, platinum, gold, titanium, tantalum, iridium, tungsten, Nitinol, or inconel. In yet another embodiment, the ring frame includes a polymer material.

In other embodiments, the valve pocket height is less than 40, 30, 15 or 10 percent of the expanded ring frame width. In yet another embodiment, the expanded ring frame forms a substantially planar structure.

In another embodiment, the artificial valve prosthesis is adapted to allow limited retrograde fluid flow.

In yet another embodiment, a portion of the support structure is adapted to expand upon deployment to create an artificial sinus in the bodily passage adjacent to the ring frame.

In another embodiment, the valve leaflet includes a material selected from a synthetic biocompatible polymer, cellulose acetate, cellulose nitrate, silicone, polyethylene, teraphthalate, polyurethane, polyamide, polyester, polyorthoester, poly anhydride, polyether sulfone, polycarbonate, polypropylene, high molecular weight polyethylene, a fluoroplastic material, polytetrafluoroethylene, or mixtures or copolymers thereof; polylactic acid, polyglycolic acid or copolymers thereof, a polyanhydride, polycaprolactone, polyhydroxy-butyrate valerate, polyhydroxyalkanoate, a polyetherurethane urea, naturally derived or synthetic collagenous material, an extracellular matrix material, submucosa, small intestinal submucosa, stomach submucosa, urinary bladder submucosa, uterine submucosa, renal capsule membrane, dura mater, pericardium, serosa, peritoneum or basement membrane materials, and liver basement membrane.

In yet another embodiment, the valve leaflet includes a bioremodelable material, for example, small intestinal submucosa.

### BRIEF DESCRIPTION OF THE DRAWINGS

Embodiments of the present invention will now be described by way of example with reference to the accompanying drawings.

FIG. 1 is an illustration depicting a cross-sectional view of a native venous valve and a retrograde blood flow pattern.

FIGS. 2(a)-(f) are illustrations depicting the interaction between valve leaflet positioning and the shape of valve pockets between the valve leaflets and the wall of a vessel. FIGS. 2(a)-(c) illustrate a valve positioned within a vessel lumen. FIGS. 2(d)-(f) illustrate a valve positioned at a sinus. The valve support structure is not shown.

FIGS. 3(a)-(f) are schematic views of an illustrative embodiments of the present invention. FIGS. 3(a)-(d) depict a monocuspid valve prosthesis having a valve leaflet attached to a ring frame. In FIGS. 3(a)-(b) the leaflet is attached to a flat ring frame. In FIGS. 3(c)-(d) the leaflet is attached to a ring frame having a shallow convex profile orientated proximally. FIGS. 3(a) and 3(c) depict the valve leaflet in an open position allowing antegrade fluid flow. FIGS. 3(b) and 3(d) depict the valve leaflet in a closed position restricting retrograde fluid flow. FIGS. 3(e)-(f) depict a valve prosthesis having portions of the perimeter of a valve leaflet attached to a ring frame at multiple positions. The valve support structure is not shown.

FIGS. 4(a)-(b) are schematic views of another illustrative embodiment of the present invention depicting a bicuspid valve prosthesis having valve leaflets attached to a ring frame having a shallow convex profile orientated proximally. FIG. 4(a) depicts the valve leaflets in an open position allowing antegrade fluid flow. FIG. 4(b) depicts the valve leaflets in a closed position restricting retrograde fluid flow. The valve support structure is not shown.

FIGS. 5(a)-(b) are schematic views of an illustrative embodiment of the present invention depicting a tricuspid valve prosthesis having valve leaflets attached to a ring frame having a shallow convex profile orientated proximally. FIG. 5(a) depicts the valve leaflets in an open position allowing antegrade fluid flow. FIG. 5(b) depicts the valve leaflets in a closed position restricting retrograde fluid flow. The valve support structure is not shown.

FIG. 6 is a schematic view of an illustrative embodiment of the present invention depicting the valve prosthesis including a support structure having interconnecting proximal and distal sections defining an intermediate, substantially open section. Two valve leaflets supported by a ring frame are positioned within the intermediate section.

FIG. 7 is a schematic view of an illustrative embodiment of the present invention depicting the valve prosthesis in which the intermediate section of the prosthesis includes an expanded portion of the support structure.

FIGS. 8(a) and 8(b) are schematic views of an illustrative embodiment of the present invention depicting a valve prosthesis allowing for limited retrograde fluid flow.

### DESCRIPTION OF THE ILLUSTRATIVE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, and alterations and modifications in the illustrated device, and further applications of the principles of the invention as illustrated therein are herein contemplated as would normally occur to one skilled in the art to which the invention relates.

Devices and systems of the invention are desirably adapted for deployment within a body lumen, and in particular embodiments, devices and systems of the invention are adapted for deployment within the venous system. Accordingly, preferred devices adapted are venous valves, for example, for percutaneous implantation within veins of the legs or feet to treat venous insufficiency.

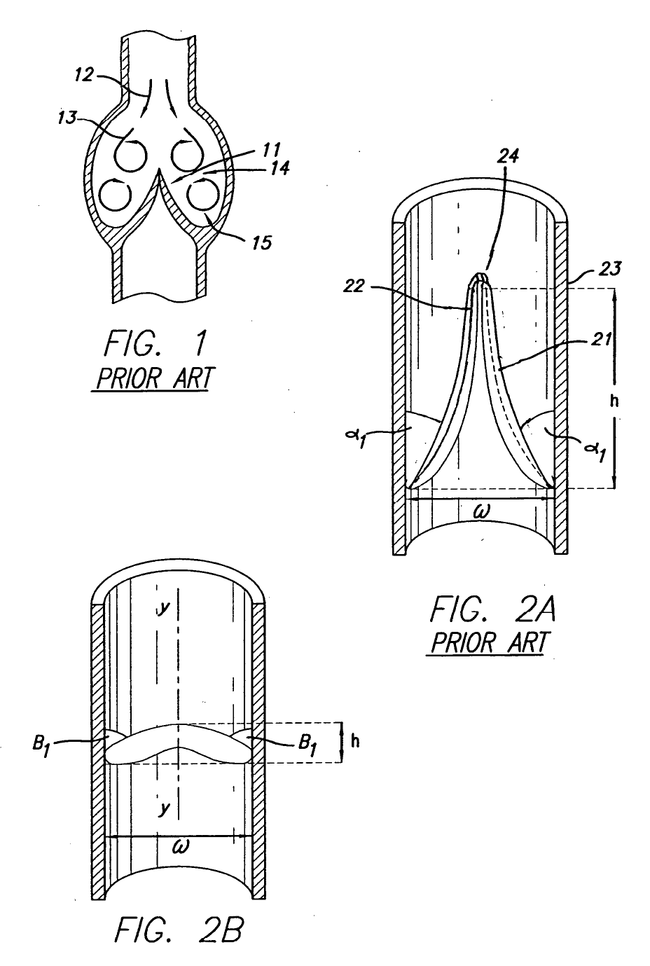

FIG. 1 illustrates a natural venous valve 11 in which retrograde blood 12 flowing or falling back down and closing the valve is thought to create a series of vortices 13 as it contacts the valve leaflets. It is believed, but not relied upon for the present invention, that the rounded shape of the enlarged natural sinus 14 surrounding the valve 11, which results in an enlargement of the valve pockets 15 between the valve leaflets and the wall of the vessel, facilitates creation of these vortices, thereby preventing blood from pooling or stagnating within the pockets 15, which may lead to thrombus formation or other problems.

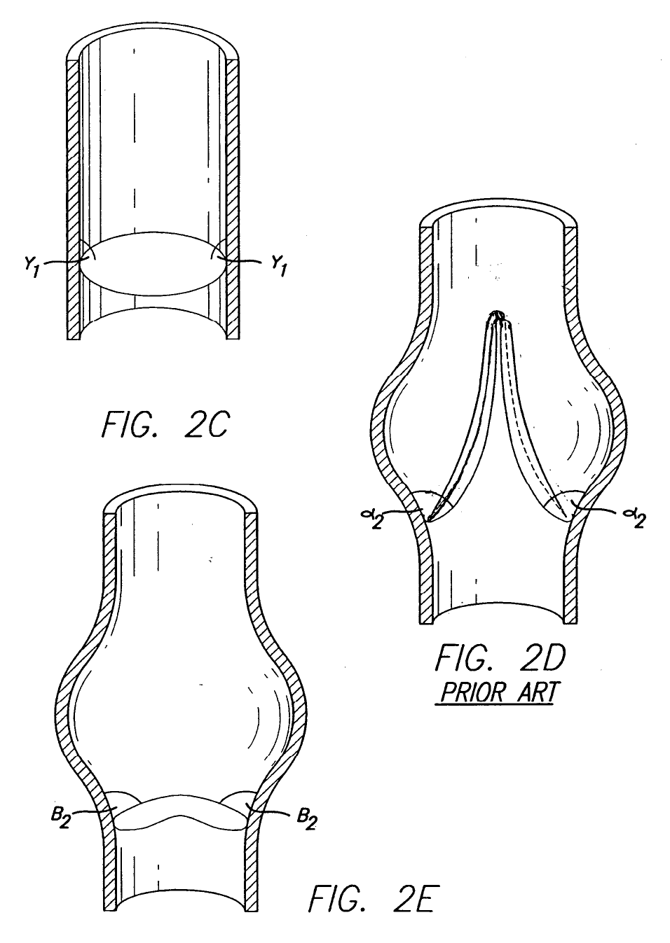

FIG. 2(a) illustrates an artificial valve prosthesis having two leaflets 21 and 22 positioned within a vessel 23. The valve leaflets are held in position by a frame and are positioned so as to meet in the vessel lumen at a position 24 proximal to the region of attachment of the valve leaflets to the vessel wall. The leaflets are positioned so that, when the leaflets are closed in response to retrograde flow, the angle α₁ between the plane of the leaflets and the vessel wall is small, typically much less than 45 degrees. This results from the supporting frame structure having a valve leaflet height greater than the valve width. Such a configuration results in the formation of narrow valve pockets when the valve leaflets are closed in response to retrograde flow.

One aspect of the present invention provides a self expanding or otherwise expandable artificial valve prosthesis for deployment within a bodily passageway, such as a vessel or duct of a patient. The prosthesis is typically delivered and implanted using well-known transcatheter techniques for self-expanding or otherwise expandable prostheses. The valve prosthesis is positioned so as to allow antegrade fluid flow and to restrict retrograde fluid flow. Antegrade fluid flow typically travels from the distal end of the prosthesis to the proximal end of the prosthesis, the latter being located closest to the heart in a venous valve when placed within the lower extremities of a patient.

The valve prosthesis includes a support structure and a valve structure. The valve structure includes a ring frame and at least one valve leaflet, a portion of the perimeter of which is attached to the ring frame. The valve leaflet is configured to deform to selectively allow fluid flow in a antegrade direction and to restrict fluid in a retrograde direction by opening or closing in response to changes in the fluid pressure differential, such as in the presence of retrograde flow. The present invention includes structural features that modify the flow dynamics within the prosthesis such that fluid collecting in pockets of the valve leaflets (leaflet pockets) is more likely to be flushed away or effectively mixed with fresher incoming bodily fluid on a continual basis.

The present invention, by virtue of the configuration of the ring frame supporting the valve leaflets, provides valve prostheses having valve pockets favorable for the formation of vortices within the valve pockets when the valve leaflets are closed in response to retrograde fluid flow within the vessel. It is also within scope of the present invention that the support structure for the ring frame is configured to create an artificial sinus to further improve the flow dynamics within the prosthesis by further broadening of the valve pocket between the valve leaflet and the vessel wall. Such configurations are depicted in FIGS. 2(e)-(f). Examples of such an artificial sinus are disclosed in copending U.S. patent application Ser. No. 10/828,716, “Artificial Valve Prosthesis with Improved Flow Dynamics”, filed Apr. 21, 2004 and published as U.S. 2004/0260389A1 on Dec. 23, 2004, the contents of which are incorporated by reference.

The Ring Frame

Embodiments of the present invention provide artificial valve prostheses having at least one valve leaflet supported by an expandable ring frame. The expanded ring frame is held in position within a vessel by a support structure. The ring frame can be manufactured separately from the support structure and attached to the support structure by methods such as welding or adhesives. Alternatively, the ring frame and the support structure can be manufactured as a single unit.

The expanded ring frame is normally positioned so that the plane of the expanded ring frame is substantially perpendicular to the distal-proximal (longitudinal) axis of the support structure. For the purposes of the invention, the plane of the expanded ring frame is considered to be “substantially perpendicular” to the distal-proximal axis of the support structure when the plane of the expanded ring frame is inclined at an angle of between 50 degrees and 150 degrees to the distal-proximal axis of the support structure. Preferably, the plane of the expanded ring frame is inclined at an angle of 90 degrees to the distal-proximal axis of the support structure.

In one illustrative embodiment, the shape of the expanded ring frame is that of an ellipsoid (“distorted ring”) having a convex/concave profile with the convex profile orientated towards the proximal end of the valve prosthesis, as is depicted in FIG. 2(b). In another illustrative embodiment, the shape of the expanded ring frame is circular, as is depicted in FIG. 2(c). Many other shapes, including ellipsoids and irregular shapes, are possible so long as the ring frame provides for the attachment and support of valve leaflet material.

The ring frame can be manufactured from a single piece of material, for example by laser cutting. Alternatively, the ring frame can be constructed from multiple separate elements physically joined together, for example, by welding.

In one embodiment, the ring frame is attached to the support structure so as to limit or prevent fluid flow between the ring frame and the vessel wall. For example, any openings between the ring frame and the vessel wall can be sealed by a covering of biologically-derived or synthetic biocompatible material, such as a collagenous extracellular matrix (e.g. SIS), pericardial tissue or fabric. Such a covering can also be attached to the support structure to assist in sealing any openings between the support structure and the vessel wall.

In general, the ring frame is dimensioned to support one or more valve leaflets in a configuration where the flow dynamics within the prosthesis are such that fluid collecting in the leaflet pockets is more likely to be flushed away or effectively mixed with fresher incoming bodily fluid on a continual basis.

In one embodiment of the present invention, such a configuration is achieved by attaching the leaflet(s) to the ring frame so that the valve pocket height is shallow compared to the width of the ring frame. For the purposes of this invention, when the expanded ring frame rests on a flat horizontal surface, the valve pocket height is the vertical distance between the lowest and highest point of attachment of a valve leaflet to the ring frame.

When the ring frame is expanded and positioned within a vessel, the valve pocket height will generally correspond to the axial distance between the most distal point of attachment of a valve leaflet on the circumference of the ring frame and the most proximal point of attachment of the valve leaflet on the circumference of the ring frame. Thus, for a “flat” spherical ring frame placed perpendicular to the axis of flow within the vessel, the valve pocket height is essentially zero. For a “distorted ring”, such as that shown in FIG. 2(b), the valve pocket height is height h measured along axis y-y.

In one embodiment, valve pocket height is less than the maximum cross-sectional dimension of the lumen defined by the expanded ring frame (the “ring frame width”). In another embodiment, the ring frame width is substantially equal to the width of the support structure at the position of attachment of the ring frame to the support structure. In other embodiments, the ring frame width is at least 95, 90, 80 70, 60, 50 or 40 percent of the width of the support structure at the attachment position.

In various embodiments, the valve pocket height is less than 45, 30, 15 or 10 percent of the expanded ring frame width. In another embodiment, the expanded ring frame forms a substantially planar structure, as is depicted in FIG. 2(c).

Illustrative Valve Prostheses

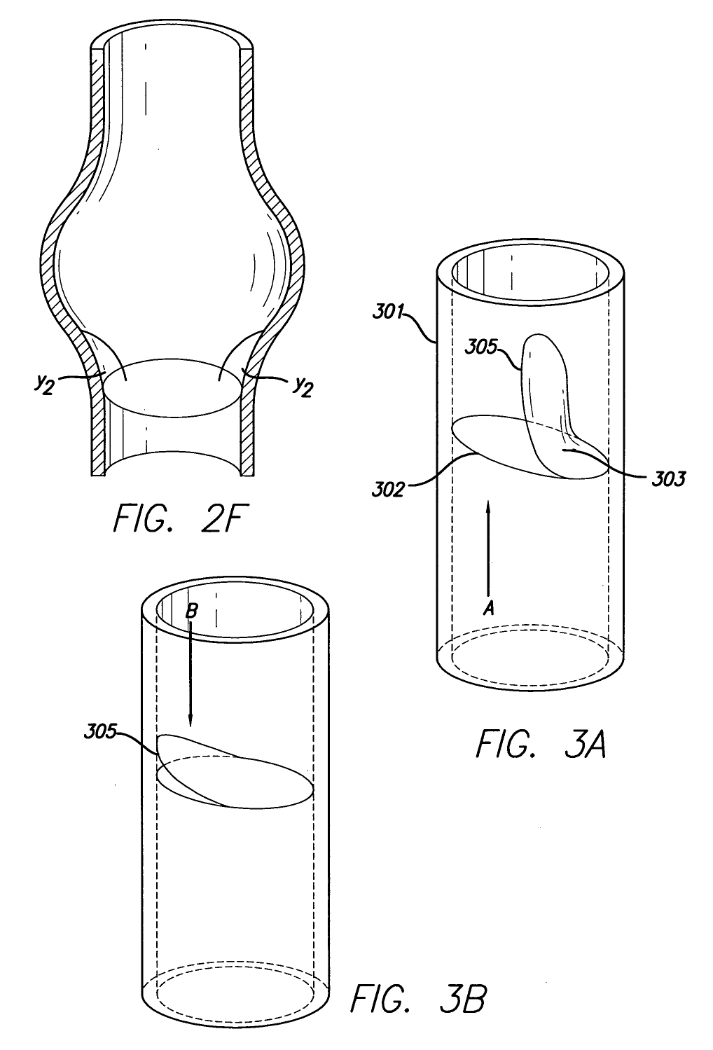

FIGS. 3(a)-(f) depict illustrative embodiments of a valve prosthesis of the present invention. FIG. 3(a) and FIG. 3(b) depict a monocuspid valve prosthesis positioned within a vessel 301. The valve prosthesis includes a valve leaflet 303 having a first portion of its perimeter attached to a ring frame 302. The ring frame 302 is positioned across the lumen of a vessel by a support structure (not shown). A second portion 305 of the perimeter of the valve leaflet 303 is not attached to the ring frame 302 and is positioned proximally of ring frame 302, i.e. downstream with respect to antegrade flow in the direction of Arrow A. In one embodiment, the valve leaflet is attached to at least 20 percent of the perimeter of the ring frame. In other embodiments, the valve leaflet is attached to at least 30, 40, 50, 60 or 70 percent of the perimeter of the ring frame.

In the embodiment illustrated in FIG. 3(a) and FIG. 3(b), the ring frame 302 has a substantially flat profile. Alternatively, as is depicted in FIG. 3(c) and FIG. 3(d), the shape of the expanded ring frame 302 is that of a “distorted ring” such that it forms a convex/concave profile with the convex profile orientated towards the proximal end of the valve prosthesis, i.e. towards the direction of antegrade fluid flow.

In the embodiment illustrated in FIG. 3(a) portions 305 of valve leaflet 303 are positioned proximally (downstream) and away from of ring frame 302 in response to fluid flow in an antegrade direction (the direction of arrow A). In FIG. 3(b), valve leaflet 303 is positioned against ring frame 302 and the wall of vessel 301 in response to flow in a retrograde direction (the direction of arrow B).

The second portion 305 of the perimeter of the valve leaflet 303 may extend beyond the perimeter of ring frame 302. In this embodiment, the valve leaflet 303 and the wall of vessel 301 form a seal when the valve leaflet is positioned to restrict flow in a retrograde direction. In one embodiment, the second portion of the valve leaflet extends beyond the perimeter of ring frame by at least 10 percent of the width of the ring frame. In other embodiments, the second portion of the valve leaflet extends beyond the perimeter of ring frame by at least 20, 30, 40, 50, 60, 80 or 100 percent of the ring frame width.

In certain embodiments, the second portion 305 or the body of valve leaflet 303 may include a stiffening member to prevent the section portion 305 from becoming positioned distally of ring frame 302. In certain other embodiments, the second portion 305 or the body of valve leaflet 303 may include attachments to a proximal region of the support structure so as to prevent the perimeter of the valve leaflet 303 becoming positioned distally of ring frame 302.

FIG. 3(e) and FIG. 3(f) depict another illustrative embodiment of the present invention. In this embodiment, portions of the perimeter of valve leaflet 303 are attached to the ring frame at multiple regions 308 and are free of the ring frame at multiple regions 309. In FIG. 3(e), fluid flow in an antegrade direction (the direction of arrow A) positions those portions of the perimeter of valve leaflet 303 that are free of the ring frame 302 proximally of and away from of ring frame 302. In FIG. 3(f), fluid flow in an retrograde direction (the direction of arrow B) positions those portions of the perimeter of valve leaflet 303 that are free of the ring frame 302 against ring frame 302 and the wall of vessel 301. In one embodiment, the valve leaflet is attached to a total of at least 20 percent of the perimeter of the ring frame. In other embodiments, the valve leaflet is attached to a total of at least 30, 40, 50, 60, 70, 80 or 90 percent of the perimeter of the ring frame.

Those portions of the perimeter of valve leaflet 303 that are free of the ring frame 302 may extend beyond the perimeter of ring frame 302 so as to assist in the formation of a seal between valve leaflet 303 and the wall of vessel 301 when valve leaflet is positioned to restrict flow in a retrograde direction and to prevent portions 309 of the perimeter of the valve leaflet 303 from becoming positioned distally of ring frame 302. In one embodiment, the free portions of the valve leaflet extend beyond the perimeter of ring frame by at least 10 percent of the width of the ring frame. In other embodiments, the free portions of the valve leaflet extend beyond the perimeter of ring frame by at least 20, 30, 40, 50, 60, 80, or 100 percent of the width of the ring frame.

Valve leaflet 303 can include a stiffening member to prevent portions of the perimeter of the valve from becoming positioned distally of ring frame 302. In certain other embodiments, portions of valve leaflet 303 may include attachments to a proximal region of the support structure so as to prevent the perimeter of the valve leaflet 303 becoming positioned distally of ring frame 302.

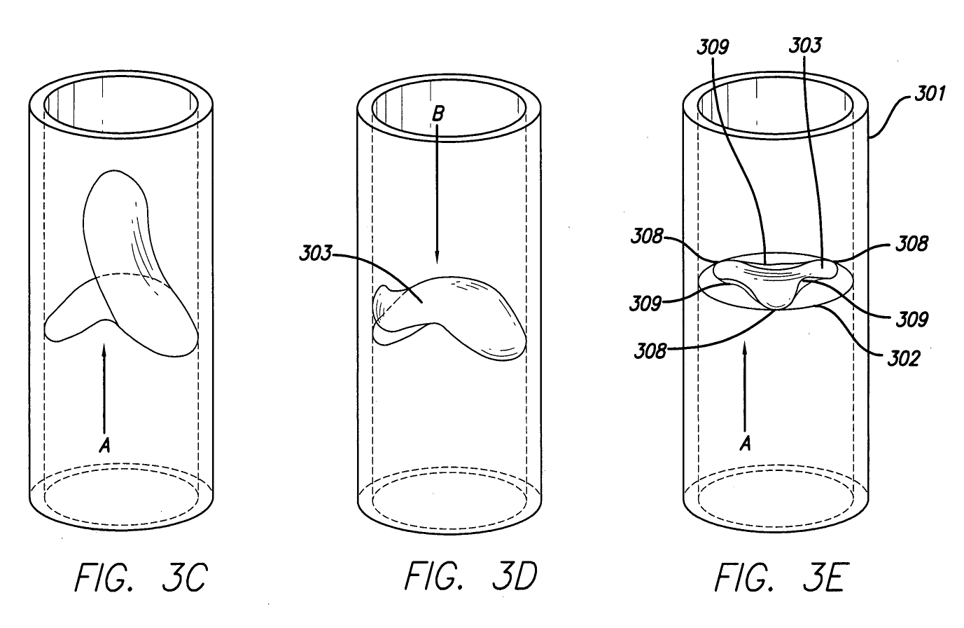

FIG. 4(a) and FIG. 4(b) depict another illustrative embodiment of the present invention. In this embodiment, two valve leaflets 403 and 407 are attached to ring frame 402, which includes two ring frame portions 404 and 405 which jointly form the ring frame. A first portion of the perimeter of the first valve leaflet 403 is attached to ring frame portion 404. A second portion 406 of the perimeter of the first valve leaflet 403 is not attached to the ring frame. A first portion of the perimeter of the second valve leaflet 407 is attached to ring frame portion 405. A second portion 408 of the perimeter of the second valve leaflet 407 is not attached to the ring frame.

When the valve is deployed within a vessel and when fluid flows in the antegrade direction, the valve leaflets position so that the free portions of the perimeter of valve leaflets 403 and 408 define a lumen allowing fluid flow in an antegrade direction (the direction of arrow A in FIG. 4(a)). When fluid flows in the retrograde direction, (the direction of arrow B in FIG. 4(b)), the leaflets 403 and 408 position so as to close the lumen, as is shown in FIG. 4(b). The portions of the perimeter of valve leaflets 403 and 407 that are not attached to the ring frame may be extended to increase the contact length 409 about the proximal portion of the valve leaflets 403 and 407 when the valve leaflets are positioned to restrict retrograde flow. Typically, the contact length is between 25 and 250 percent of the vessel diameter. In certain embodiments, the contact length is between 25 and 200 percent of the vessel diameter. In certain other embodiments, the contact length is between 25 and 150 percent of the vessel diameter.

The amount of slack in the valve leaflet material also helps determine how well the valve leaflets coapt during retrograde flow and how large of an opening they permit during antegrade flow. In one embodiment, the valve prosthesis is configured such that the distance formed between the leaflets in their fully open position remains between 0-100 percent of the width of the ring frame. In another embodiment, the valve prosthesis is configured such that the distance remains between 20-80% of the width of the ring frame. In yet another embodiment, the valve prosthesis is configured such that the distance remains between 50-70% of the width of the ring frame.

In general, the shape of the ring frame results in the enlargement of the valve pockets 410 between the valve leaflets 403 and 407 and the vessel wall 401. This configuration facilitates the creation of vortices, resulting in a reduction in pooling of blood when the valve leaflets 403 and 407 are closed in response to retrograde fluid flow.

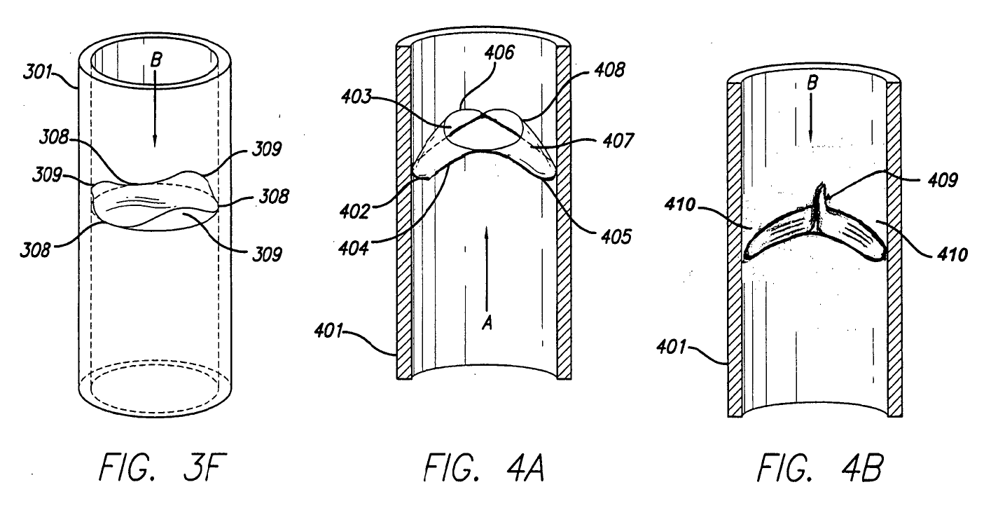

FIG. 5(a) and FIG. 5(b) depict another illustrative embodiment of an artificial valve prosthesis of the present invention. In this embodiment, the ring frame is formed from three portions 503, 504, and 505 joined to form a continuous perimeter. Portions of the perimeter of each of valve leaflets 506, 507 and 508 are attached to the perimeter of portions 503, 504, and 505 respectively. Other portions 509, 510, and 511 of the perimeter of valve leaflets are not attached to the ring frame.

FIG. 5(a) illustrates the configuration of the valve leaflets when the valve prosthesis is subjected to antegrade fluid flow (i.e. in the direction of arrow A). In this configuration, antegrade fluid flow positions the unattached portions of the perimeter of the leaflets to define a lumen. FIG. 5(b) shows the configuration of the leaflets 506, 507, and 508 when the valve prosthesis is subjected to retrograde fluid flow (i.e. in the direction of arrow B). In this configuration, retrograde fluid flow positions the valve leaflets so that the unattached portions of the perimeter to the valve leaflets contact with each other to close the lumen. Portions 509, 510, and 511 may be extended, as is described above, to increase the contact length of the proximal portions of the valve leaflets.

It will be understood that other valve body configurations are also contemplated as being within the scope of the present invention. For example, valves having four (quadracuspid valve), or more leaflets, are contemplated. Hence, the number of leaflets possible for embodiments of the present invention can be one, two, three, four, or any practical number, but bi-leaflet valves may prove advantageous in low-flow venous situation as compared to tri-leaflet embodiments, such the type used as heart valves.

Valve Support Structure

The support structure can be, for example, formed from wire, cut from a section of cannula, molded or fabricated from a polymer, biomaterial, or composite material, or a combination thereof. The pattern (i.e., configuration of struts and cells) of the anchoring portion(s) that is selected to provide radial expandability to the prosthesis is also not critical for an understanding of the invention. Any support structure is applicable for use with the claimed valve prosthesis so long as this structure supports the ring frame in the required position. Numerous examples of support structures are disclosed in copending patent U.S. patent application Ser. No. 10/642,372 entitled, Implantable Vascular Device, filed Aug. 15, 2003, the contents of which are incorporated by reference.

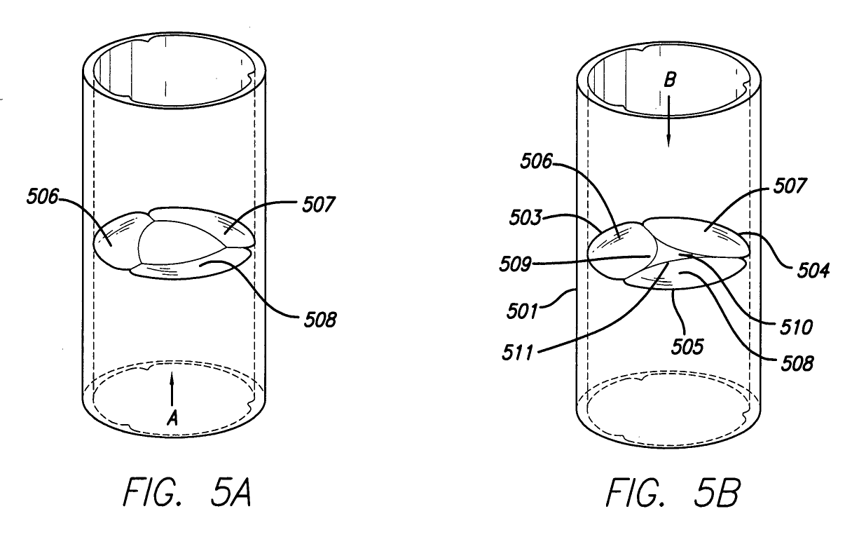

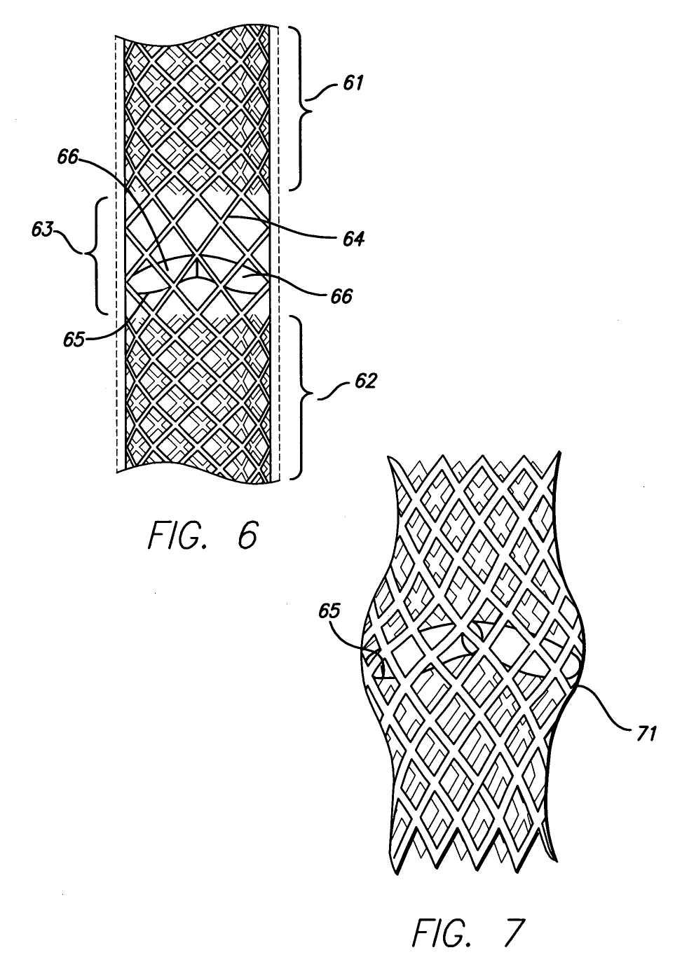

FIG. 6 and FIG. 7 illustrate embodiments in which the valve prosthesis includes a support structure having a first section 61 and a second section 62 that are spaced apart from one another, defining an intermediate section 63 containing the ring frame 65 and attached valve leaflets 66. Sections 61 and 62, which preferably comprise a pair of radially expandable or self-expanding anchoring portions, are joined by an interconnecting means, such as the illustrative pair of connection struts 64, which also support ring frame 65. In the embodiments of the present invention, the anchoring portions may function as stents to help the bodily passage remain open, but their primary function is limited to engaging the bodily passage to support ring frame 65.

In certain embodiments, the intermediate section 63 is a substantially open section creating an artificial sinus on the vessel. The term “substantially open section” is used herein to define a largely unsupported portion of the bodily passage in which at least some minimal interconnecting structure (e.g., thin or flexible elements aligned with the leaflet commissures) is present that traverses the unsupported portion of the bodily passage, but that comprises very limited surface area and typically supplies minimal, if any, force against the walls of the passageway lateral to the valve prosthesis.

Sections 61 and 62 generally assume a fixed diameter after deployment. The intermediate section, which is substantially open, expands to form a bulging region of the vessel that functions as an artificial sinus. Further details concerning the construction of support structures having intermediate regions adapted for the formation of an artificial sinus can be found in co-pending patent application Ser. No. 10/828,716, the contents of which are incorporated by reference.

In the illustrative embodiment depicted in FIG. 6, the ring frame 65 supporting a pair of leaflets 66 is situated in the intermediate section and attached to the proximal section 61 and distal section 62 of the support structure. The valve prosthesis is configured so that it advantageously expands with the deployment of the proximal and distal sections 61 and 62 and ring frame 65 such that the outer edges of ring frame 65 contact the vessel wall sufficiently to at least substantially prevent leakage of bodily fluid around the valve structure.

In another embodiment, depicted in FIG. 7, the support structure includes an expanded portion 71, larger in diameter than the remainder of the support structure, and that upon deployment, creates an artificial sinus surrounding the ring frame 65.

Controlled Retrograde Flow

The artificial valve prosthesis of the present invention can be configured to permit a controlled amount of retrograde flow through a body vessel despite the presence of the valve prosthesis. This may be desirable for a variety of reasons. For example, allowance of a controlled amount of retrograde flow can assist in the prevention of pooling of fluid when the valve prosthesis is in a closed or substantially closed configuration in the body vessel.

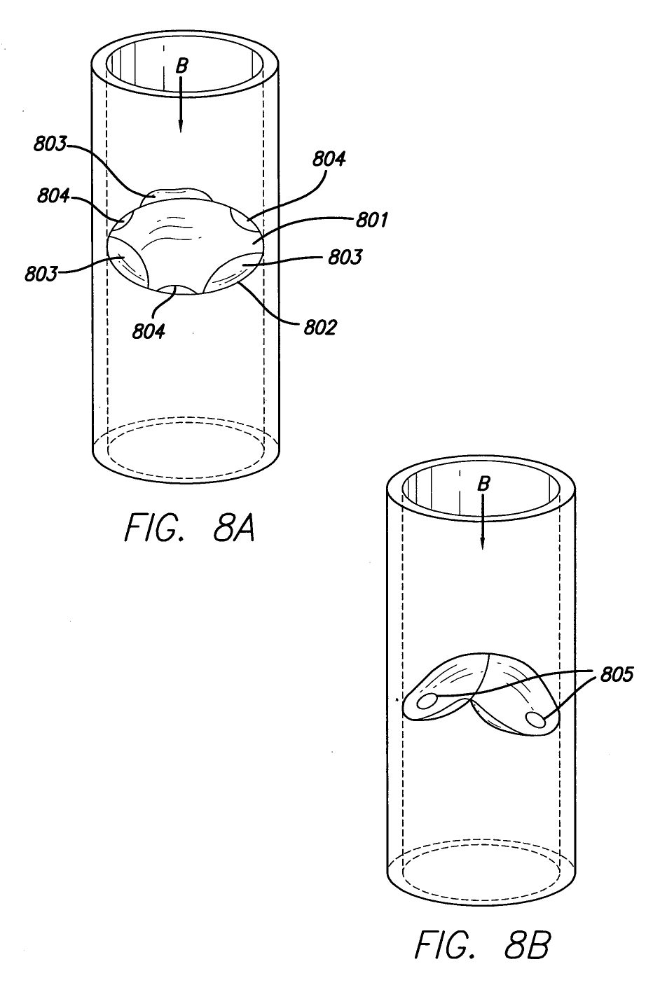

Any suitable means for permitting a controlled amount of retrograde flow to pass through the valve prosthesis can be used in any of the embodiments described herein. FIG. 8 illustrates embodiments of an artificial valve prosthesis that includes suitable means for permitting a controlled amount of retrograde flow. In the embodiment depicted in FIG. 8(a), the valve prosthesis is positioned within a vessel to restrict retrograde flow in the vessel. Regions of the valve leaflet perimeter 803 are free of ring frame 802 and can extend beyond the perimeter of ring frame 802. Retrograde flow positions regions 803 against ring frame 802 and the vessel wall so as to restrict retrograde flow. Portions of the perimeter of the valve leaflet 804 are not attached to ring frame 802 and do not extend beyond the perimeter of the ring frame. When subjected to retrograde flow, gaps are formed between the perimeter of the valve leaflet 804 and ring frame 802. These gaps allow limited retrograde flow. FIG. 8(b) depicts an alternative embodiment in which apertures 805 are present in the body of the valve leaflets.

The quantity of retrograde flow that passes through the aperture is controlled by the overall dimensions and configuration of the aperture. A larger lumen allows a greater amount of retrograde flow to pass through the valve prosthesis while a relatively smaller lumen will allow a relatively lesser amount of retrograde flow to pass. The dimensions and configuration of the aperture of each embodiment can be optimized based upon the vessel in which the valve prosthesis is placed. The size and configuration selected will depend on several factors, including the vessel size, typical flow volumes and rates, and others. The lumen is advantageously sized to allow a desired amount of retrograde flow pass through the lumen during periods of retrograde flow. The aperture should be small enough, though, to still allow the valve prosthesis to substantially prevent retrograde flow when the valve prosthesis is in a closed configuration.

Thus, the aperture is advantageously sized so as to not allow a majority of retrograde flow to pass through the aperture. In one embodiment, the total open area of the aperture is, at a maximum, less than the cross-sectional area of the vessel lumen. As used herein, the term “total open area”, in relation to the aperture, refers to the total area of the aperture when the entire perimeter of the aperture lies in the same plane.

The aperture advantageously can be sized to mimic the degree of retrograde flow—the leakiness—that is present in a natural valve located at the point of treatment in the body vessel. Accordingly, the dimensions of the aperture can be determined and optimized based upon the vessel in which the frameless grafting prosthesis is to be placed. For venous valve applications, the total open area of the aperture is advantageously less than about 50% of the cross-sectional area of the vessel at the intended point of deployment. More advantageously, the total open area of the aperture is less than about 25% of the total cross-sectional area of the vessel at the intended point of deployment. In one example, a device is configured for placement in a vessel having a total cross-sectional area of about 50 mm². In this example, the aperture has a total open area of about 20 mm². Also for venous valve applications, a circular lumen with a diameter of between about 0.5 and about 3.0 mm has been found to be suitable. In a specific venous valve example, a circular lumen with a diameter of about 1 mm has been found to be suitable. In another specific venous valve example, a circular lumen with a diameter of about 2 mm has been found to be suitable.

The aperture can have any suitable shape. Examples of specifically contemplated shapes include circular, ovoid, triangular, square, rectangular, and tear-drop shaped openings. Furthermore, multiple openings can be used. In these embodiments, the sum total open area of all openings is advantageously in accordance with the parameters described above. Further examples of valves having apertures allowing limited retrograde flow are disclosed in U.S. 2004/0225352A1, published Nov. 11, 2004, the contents of which are incorporated by reference.

Support Structure and Ring Frame Composition

It should be understood that the materials used in the support structure and/or the ring frame can be selected from a well-known list of suitable metals and polymeric materials appropriate for the particular application, depending on necessary characteristics that are required (self-expansion, high radial force, collapsibility, etc.). Suitable metals or metal alloys include: stainless steels (e.g., 316, 316L or 304), nickel-titanium alloys including shape memory or superelastic types (e.g., nitinol or elastinite); inconel; noble metals including copper, silver, gold, platinum, paladium and iridium; refractory metals including Molybdenum, Tungsten, Tantalum, Titanium, Rhenium, or Niobium; stainless steels alloyed with noble and/or refractory metals; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); L605; magnetic ferrite; bioabsorbable materials, including magnesium; or other biocompatible metals and/or alloys thereof.

In various embodiments, the ring frame comprises a metallic material selected from stainless steel, nickel, silver, platinum, gold, titanium, tantalum, iridium, tungsten, a self-expanding nickel-titanium alloy, NITINOL, or inconel.

One particularly preferred material for forming a frame is a self-expanding material such as the superelastic nickel-titanium alloy sold under the tradename NITINOL. Materials having superelastic properties generally have at least two phases: a martensitic phase, which has a relatively low tensile strength and which is stable at relatively low temperatures, and an austenitic phase, which has a relatively high tensile strength and which can be stable at temperatures higher than the martensitic phase. Shape memory alloys undergo a transition between an austenitic phase and a martensitic phase at certain temperatures. When they are deformed while in the martensitic phase, they retain this deformation as long as they remain in the same phase, but revert to their original configuration when they are heated to a transition temperature, at which time they transform to their austenitic phase. The temperatures at which these transitions occur are affected by the nature of the alloy and the condition of the material. Nickel-titanium-based alloys (NiTi), wherein the transition temperature is slightly lower than body temperature, are preferred for the present invention. It can be desirable to have the transition temperature set at just below body temperature to insure a rapid transition from the martinsitic state to the austenitic state when the frame can be implanted in a body lumen.

Preferably, the ring frame comprises a self-expanding nickel titanium (NiTi) alloy material. The nickel titanium alloy sold under the tradename NITINOL is a suitable self-expanding material that can be deformed by collapsing the frame and creating stress which causes the NiTi to reversibly change to the martensitic phase. The frame can be restrained in the deformed condition inside a delivery sheath typically to facilitate the insertion into a patient's body, with such deformation causing the isothermal phase transformation. Once within the body lumen, the restraint on the frame can be removed, thereby reducing the stress thereon so that the superelastic frame returns towards its original undeformed shape through isothermal transformation back to the austenitic phase. Other shape memory materials may also be utilized, such as, but not limited to, irradiated memory polymers such as autocrosslinkable high density polyethylene (HDPEX). Shape memory alloys are known in the art and are discussed in, for example, “Shape Memory Alloys,” Scientific American, 281: 74-82 (November 1979), incorporated herein by reference.

Some embodiments provide frames that are not self-expanding, or that do not comprise superelastic materials. For example, in other embodiments, the frame can comprise silicon-carbide (SiC). For example, published U.S. Patent Application No. US2004/034409 to Hueblein et al., published on Feb. 14, 2004 and incorporated in its entirety herein by reference, discloses various suitable frame materials and configurations.

Other suitable materials used in the support structure and/or the ring frame include carbon or carbon fiber; cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene,
polytetrafluoroethylene, or another biocompatible polymeric material, or mixtures or copolymers of these; polylactic acid, polyglycolic acid or copolymers thereof, a polyanhydride, polycaprolactone,
polyhydroxybutyrate valerate or another biodegradable polymer, or mixtures or copolymers of these; a protein, an extracellular matrix component, collagen, fibrin or another biologic agent; or a suitable mixture of any of these.

Also provided are embodiments wherein the support structure and/or ring frame comprises a means for orienting the frame within a body lumen. For example, the frame can comprise a marker, such as a radiopaque portion that would be seen by remote imaging methods including X-ray, ultrasound, Magnetic Resonance Imaging and the like, or by detecting a signal from or corresponding to the marker. In other embodiments, indicia can be located, for example, on a portion of a delivery catheter that can be correlated to the location of the support structure and/or ring frame within a body vessel. The addition of radiopacifiers (i.e., radiopaque materials) to facilitate tracking and positioning of the medical device may be added in any fabrication method or absorbed into or sprayed onto the surface of part or all of the medical device. The degree of radiopacity contrast can be altered by implant content. Radiopacity may be imparted by covalently binding iodine to the polymer monomeric building blocks of the elements of the implant. Common radiopaque materials include barium sulfate, bismuth subcarbonate, and zirconium dioxide. Other radiopaque elements include: cadmium, tungsten, gold, tantalum, bismuth, platinum, iridium, and rhodium. Radiopacity is typically determined by fluoroscope or x-ray film.

Valve Leaflet Composition

The material used in body of the valve leaflet includes a biocompatible material, and is, in one embodiment, a bioremodelable material. Suitable bioremodelable materials may be made from natural or synthetic polymers, including collagen. Thus, in general, the flexible material may comprise a synthetic biocompatible polymer such as cellulose acetate, cellulose nitrate, silicone, polyethylene, teraphthalate, polyurethane, polyamide, polyester, polyorthoester, poly anhydride, polyether sulfone, polycarbonate, polypropylene, high molecular weight polyethylene, a fluoroplastic material such as polytetrafluoroethylene, or mixtures or copolymers thereof; polylactic acid, polyglycolic acid or copolymers thereof, a polyanhydride, polycaprolactone, polyhydroxy-butyrate valerate, polyhydroxyalkanoate, or another biodegradable polymer.

In certain embodiments of the invention, the flexible material is comprised of a naturally derived or synthetic collagenous material, and especially an extracellular collagen matrix material. Suitable extracellular collagen matrix materials (“ECM material”) include, for instance, submucosa (including, for example, small intestinal submucosa (“SIS”), stomach submucosa, urinary bladder submucosa, or uterine submucosa), renal capsule membrane, dura mater, pericardium, serosa, and peritoneum or basement membrane materials, including liver basement membrane. These layers may be isolated and used as intact natural sheet forms, or reconstituted collagen layers including collagen derived from these materials or other collagenous materials may be used. For additional information as to submucosa materials useful in the present invention, and their isolation and treatment, reference can be made to U.S. Pat. Nos. 4,902,508, 5,554,389, 5,993,844, 6,206,931, and 6,099,567, the contents of which are incorporated by reference. Renal capsule tissue can also be obtained from warm blooded vertebrates, as described more particularly in copending U.S. patent application Ser. No. 10/186,150, filed Jun. 28, 2002, and International Patent Application Serial Number PCT/US02/20499, filed Jun. 28, 2002, and published Jan. 9, 2003 as International Publication Number W003002165, the contents of which are incorporated by reference.

In one embodiment of the invention, the ECM material is porcine SIS. SIS can be prepared according to the method disclosed in U.S. 2004/0180042A1, published Sep. 16, 2004, the contents of which are incorporated by reference.

In certain embodiments of the invention, the flexible material is a polyetherurethane urea. One example of a biocompatible polyurethane is THORALON (THORATEC, Pleasanton, Calif.), as described in U.S. Pat. Application Publication No. 2002/0065552 A1 and U.S. Pat. No. 4,675,361, both of which are incorporated herein by reference. According to these patents, THORALON is a polyurethane base polymer (referred to as BPS-215) blended with a siloxane containing surface modifying additive (referred to as SMA-300). Base polymers containing urea linkages can also be used. The concentration of the surface modifying additive may be in the range of 0.5% to 5% by weight of the base polymer.

The SMA-300 component (THORATEC) is a polyurethane comprising polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON can be manipulated to provide either porous or non-porous THORALON. Porous THORALON can be formed by mixing the polyetherurethane urea (BPS-215), the surface modifying additive (SMA-300) and a particulate substance in a solvent. The particulate may be any of a variety of different particulates or pore forming agents, including inorganic salts. Preferably the particulate is insoluble in the solvent. The solvent may include dimethyl formamide (DMF), tetrahydrofuran (THF), dimethyacetamide (DMAC), dimethyl sulfoxide (DMSO), or mixtures thereof. The composition can contain from about 5 wt % to about 40 wt % polymer, and different levels of polymer within the range can be used to fine tune the viscosity needed for a given process. The composition can contain less than 5 wt % polymer for some spray application embodiments. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water for the extraction, for example water at a temperature of about 60° C. The resulting pore diameter can also be substantially equal to the diameter of the salt grains.

The porous polymeric sheet can have a void-to-volume ratio from about 0.40 to about 0.90. Preferably the void-to-volume ratio is from about 0.65 to about 0.80. The resulting void-to-volume ratio can be substantially equal to the ratio of salt volume to the volume of the polymer plus the salt. Void-to-volume ratio is defined as the volume of the pores divided by the total volume of the polymeric layer including the volume of the pores. The void-to-volume ratio can be measured using the protocol described in AAMI (Association for the Advancement of Medical Instrumentation) VP20-1994, Cardiovascular Implants—Vascular Prosthesis section 8.2.1.2, Method for Gravimetric Determination of Porosity. The pores in the polymer can have an average pore diameter from about 1 micron to about 400 microns. Preferably the average pore diameter is from about 1 micron to about 100 microns, and more preferably is from about 1 micron to about 10 microns. The average pore diameter is measured based on images from a scanning electron microscope (SEM). Formation of porous THORALON is described, for example, in U.S. Pat. No. 6,752,826 and 2003/0149471 A1, both of which are incorporated herein by reference.

Non-porous THORALON can be formed by mixing the polyetherurethane urea (BPS-215) and the surface modifying additive (SMA-300) in a solvent, such as dimethyl formamide (DMF), tetrahydrofuran (THF), dimethyacetamide (DMAC), dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments. The entire composition can be cast as a sheet, or coated onto an article such as a mandrel or a mold. In one example, the composition can be dried to remove the solvent.

THORALON has been used in certain vascular applications and is characterized by thromboresistance, high tensile strength, low water absorption, low critical surface tension, and good flex life. THORALON is believed to be biostable and to be useful in vivo in long term blood contacting applications requiring biostability and leak resistance. Because of its flexibility, THORALON is useful in larger vessels, such as the abdominal aorta, where elasticity and compliance is beneficial.

A variety of other biocompatible polyurethanes/polycarbamates and urea linkages (hereinafter “—C(O)N or CON type polymers”) may also be employed. These include CON type polymers that preferably include a soft segment and a hard segment. The segments can be combined as copolymers or as blends. For example, CON type polymers with soft segments such as PTMO, polyethylene oxide, polypropylene oxide, polycarbonate, polyolefin, polysiloxane (i.e. polydimethylsiloxane), and other polyether soft segments made from higher homologous series of diols may be used. Mixtures of any of the soft segments may also be used. The soft segments also may have either alcohol end groups or amine end groups. The molecular weight of the soft segments may vary from about 500 to about 5,000 g/mole.

Preferably, the hard segment is formed from a diisocyanate and diamine. The diisocyanate may be represented by the formula OCN—R—NCO, where —R— may be aliphatic, aromatic, cycloaliphatic or a mixture of aliphatic and aromatic moieties. Examples of diisocyanates include MDl, tetramethylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines contaning both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

Other applicable biocompatible polyurethanes include those using a polyol as a component of the hard segment. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof.

Biocompatible CON type polymers modified with cationic, anionic and aliphatic side chains may also be used. See, for example, U.S. Pat. No. 5,017,664.

Other biocompatible CON type polymers include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.).

Other biocompatible CON type polymers can include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible CON type polymers may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

In certain embodiments of the invention, the valve leaflet may include a stiffening member, for example, to prevent or help prevent the valve leaflet becoming positioned distally of the ring frame. As used herein, a stiffening member is a region of the valve leaflet that is less flexible than other portions of the valve leaflet. Examples of such a stiffening member include a region of increased thickness created, for example, by folding, rolling, or otherwise gathering and securing material of the valve leaflet. Alternatively, stiffening members can be formed by molding the stiffening member to have an increased thickness relative to the remainder of the body of the valve leaflet. The stiffening member may also be formed by cross linking the material comprising the stiffening member where the stiffening member is made of collagenous materials. In other embodiments, the regions of the valve leaflet may include a material, such as biocompatible metal or polymer, which is less flexible that the material used in other regions of the body of the valve leaflet. Further examples of valve leaflets having a stiffening member can be found in U.S. patent application Ser. No. 11/435,057, filed May 16, 2006, the contents of which are incorporated by reference.

Attachment of the Valve Leaflet to the Ring Frame

Methods for attaching a valve leaflet to the ring frame are also provided. The valve leaflet material can be attached to the ring frame by any appropriate attachment means, including but not limited to, adhesive, fasteners, and tissue welding using heat and/or pressure. Alternatively, the valve leaflet may be formed on the ring frame by an appropriate means, including but not limited to, spraying, electrostsatic deposition, ultrasonic deposition, or dipping.

In one embodiment of the invention, the valve prosthesis includes a valve leaflet formed from a non-porous biocompatible polyurethane based polymer such as non-porous THORALON. According to one method of attachment, a solution comprising a dissolved THORALON is coated and dried on a mandril to form a valve leaflet.

A solution for forming non-porous THORALON can be made by mixing the polyetherurethane urea (BPS-215) and the surface modifying additive (SMA-300) in a solvent, such as dimethyl formamide (DMF), tetrahydrofuran (THF), dimethyacetamide (DMAC), or dimethyl sulfoxide (DMSO). The composition can contain from about 5 wt % to about 40 wt % polymer, and different levels of polymer within the range can be used to fine tune the viscosity needed for a given process. The composition can contain less than 5 wt % polymer for some spray application embodiments.

The entire composition can be cast as a sheet, or coated onto an article such as a mandril or a mold. In one example, the composition can be dried to remove the solvent. The mandril can be made from any suitable material that permits the THORALON to coated, dried on and removed from the mandril surface. Suitable materials include stainless steel and glass. In one embodiment, at least a portion of the outer surface of the mandril is formed in the desired shape of a valve leaflet. The valve leaflet can be formed by coating a thin layer of a solution of THORALON onto the shaped portion of the mandril, drying the coating of the THORALON on the mandril surface, and carefully removing the dried layer of THORALON.

Methods of manufacturing implantable valves comprising one or more leaflets attached to a support frame are also provided. One or more valve leaflets can be attached to a support frame by any suitable technique. In one embodiment, the valve leaflets comprise THORALON that is attached to the ring frame by being formed around and encapsulating portions of the ring frame. In one method, a solution comprising dissolved THORALON is sprayed and dried on an assembly formed by fitting a ring frame over a mandril to form a valve prosthesis comprising one or more valve leaflets.

In one embodiment, one or more pre-coating layer(s) of THORALON are coated onto at least a portion of the mandril. Next, the ring frame is fitted onto the mandril. The ring frame can be any of those described above. Third, a solution comprising a DMAC solution of non-porous THORALON is coated onto the assembly comprising the mandril and the ring frame using any suitable method, including spraying or dipping.

In one embodiment, a solution of THORALON is sprayed from a spray gun onto the assembly and the mandril is rotated during spraying process to promote uniform coating of the mandril. Any suitable rate of rotation can be used that provides for a uniform coating of the mandril and retains the coated material on the surface of the mandril. In one embodiment, the mandril is rotated at a rate of about 1 rpm.

When a pre-coating layer is present on the mandril, the THORALON adheres to the pre-coating layer as the solution of THORALON is spray coated onto the surface of the assembly and forms a sheet of THORALON that encapsulates portions of the ring frame. Optionally, one or more bioactive agents can be coated onto the mandril with the THORALON.

In one embodiment, the pre-coating layer is first dried on the mandril, then the ring frame is placed over the coated mandril, and finally second layer of THORALON is spray coated over the ring frame as a solution comprising a suitable solvent such as DMAC and THORALON. The solvent in the spray solution preferably partially solubilizes the pre-coating layer so that one fused layer of THORALON is formed. The fused layer can encapsulate portions of the ring frame and be solidified by evaporation of residual solvent, thereby joining the THORALON to the ring frame. The residual solvent in the fused layer can be evaporated by heating the valve prosthesis on the mandril.

Alternatively, one or more valve leaflets can be attached to the ring frame by other methods. In one embodiment, a sheet of material is cut to form a leaflet and the edges of the leaflet are wrapped around portions of a ring frame and portions of the valve leaflet sealibly connected together to fasten the valve leaflet around the ring frame. For example, one edge of a sheet of valve leaflet material can be wrapped around a portion of the ring frame and held against the body of the valve leaflet, so that the valve leaflet material forms a lumen enclosing a portion of the ring frame. A small amount of a suitable solvent is then applied to the edge of the valve leaflet material to dissolve the edge into an adjacent portion of the valve leaflet material and thereby seal the material around the ring frame.

In another embodiment, the sheet of valve leaflet material is shaped to form the valve leaflet that is attached to a portion of a ring frame using stitching through the valve leaflet material and around a portion of the ring frame, adhesives, tissue welding or cross linking to directly join the valve leaflet material to the frame. A valve leaflet attached to a ring frame can be permitted to move relative to the ring frame, or the valve leaflet can be substantially fixed in its position or orientation with respect to the ring frame by using attachment configurations that resist relative movement of the valve leaflet and the ring frame.

An electrostatic spray deposition (ESD) method of coating the valve leaflet material onto a mandril can also be used to form a valve leaflet. In this embodiment, particles in the sprayed solution of valve leaflet material are electrostatically charged when leaving the nozzle of the spray gun and the mandril is maintained at an electrical potential or grounded to attract the charged particles from the sprayed solution of valve leaflet material. The solution of valve leaflet material is first dissolved in a solvent and then sprayed onto the mandril using an ESD process.

The ESD process generally depends on the principle that a charged particle is attracted towards a grounded target. Without being confined to any theory, the typical ESD process may be described as follows. The solution that is to be deposited on the mandril is typically charged to several thousand volts (typically negative) and the mandril held at ground potential. The charge of the solution is generally great enough to cause the solution to jump across an air gap of several inches before landing on the target. As the solution is in transit towards the target, it fans out in a conical pattern which aids in a more uniform coating. In addition to the conical spray shape, the charged particles are further attracted towards the conducting portions of the target, rather than towards any non-conductive region of the target, leaving the coating mainly on the conducting regions of the target.

Generally, the ESD method allows for control of the coating composition and surface morphology of the deposited coating. In particular, the morphology of the deposited coating may be controlled by appropriate selection of the ESD parameters, as set forth in WO 03/006180 (Electrostatic Spray Deposition (ESD) of biocompatible coatings on Metallic Substrates), the contents of which are incorporated by reference. For example, a coating having a uniform thickness and grain size, as well as a smooth surface, may be obtained by controlling deposition conditions such as deposition temperature, spraying rate, precursor solution, and bias voltage between the spray nozzle and the medical device being coated. The deposition of porous coatings is also possible with the ESD method.

One hypothetical example of an electrostatic spraying apparatus and method is provided. Specifically, a solution of a non-porous THORALON material could be loaded into a 20 mL syringe of an ESD apparatus from Teronics Development Corp., which can then be mounted onto a syringe pump and connected to a tub that carries the solution to a spray head. The syringe pump could then used to purge the air from the solution line and prime the line and spray nozzle with solution. An electrical connection to the nozzle could supply the required voltage. An electrical connection could be provided to hold the mandril at grounding potential.

A motor could then be activated to rotate the mandril at a constant speed of about 1 rpm. The syringe pump could then be activated to supply the nozzle with a consistent flow of solution, and the power supply could be activated to provide a charge to the solution and cause the solution to jump the air gap and land on the mandril surface. As the coated surface is rotated away from the spray path, the volatile portion of the solution could be evaporated leaving a coating of THORALON behind. The mandril could be continually rotated in the spray pattern until the desired amount of non-porous THORALON material accumulates. During the coating process, the mandril could preferably be kept at ambient temperature and humidity, the solution could be pumped at a rate of about 2-4 cm³/hr through the spray gun (which can be placed at a horizontal distance of approximately 6 cm from the mandril), and the bias voltage between the spray nozzle and the mandril should be approximately 10-17 kilovolts.

A ring frame could then be slipped over a mandril (Teronics Development Corp., 2 mm×30 mm) so that at least a portion of the ring frame makes an electrical connection with the mandril. The mandril could again be continually rotated in the spray pattern until the desired amount of non-porous THORALON material accumulates.

Where it is desired that portions of the perimeter of the valve leaflet material are not attached to the ring frame, the valve leaflet material may be cut to free the material from the ring frame. Alternatively, a mask may be used to cover portions of the ring frame to prevent attachment of THORALON. The mask can be made from any suitable material that permits the THORALON to coated, dried on and removed from the mask surface. In one embodiment, a mask could be applied to the mandril surface before application of pre-coating layer(s) of THORALON. After the pre-coating layer(s) are applied, the mask could be removed and the ring frame placed on the mandril. The mandril could again be continually rotated in the spray pattern until the desired amount of non-porous THORALON material accumulates. Only those portions of the ring frame placed over portions of the mandril having a pre-coating of THORALON would be enclosed in THORALON.

Further examples of methods of preparation of valve prostheses, including methods of attaching a valve leaflet to a support frame, can be found in copending patent application attorney reference number PA-5674-PRV (8627/654), entitled: Implantable Thromboresistant Valve, filed Jul. 28, 2005, Inventors: Charles W. Agnew, James D. Purdy, Jr., Brian Case and Ram H. Paul.

Bioactive Agents

Valve prosthesis of the present invention can include a bioactive agent. A bioactive agent can be included in any suitable part of the valve prosthesis, for example in the ring frame, the support structure and/or the valve leaflet. Selection of the type of bioactive agent, the portions of the valve prosthesis comprising the bioactive agent, and the manner of attaching the bioactive agent to the valve prosthesis can be chosen to perform a desired therapeutic function upon implantation.

For example, a therapeutic bioactive agent can be combined with a biocompatible polyurethane, impregnated in an extracellular collagen matrix material, incorporated in the support structure or coated over any portion of the valve prosthesis. In one embodiment, the valve prosthesis can comprise one or more valve leaflets comprising a bioactive agent coated on the surface of the valve leaflet or impregnated in the valve leaflet. In another aspect, a bioactive material is combined with a biodegradable polymer to form a portion of the support structure.

A bioactive agent can be incorporated in or applied to portions of the valve prosthesis by any suitable method that permits adequate retention of the bioactive agent material and the effectiveness thereof for an intended purpose upon implantation in the body vessel. The configuration of the bioactive agent on or in the valve prosthesis will depend in part on the desired rate of elution for the bioactive agent. Bioactive agents can be coated directly on the valve prosthesis surface or can be adhered to a valve prosthesis surface by means of a coating. For example, a bioactive agent can be blended with a polymer and spray or dip coated on the valve prosthesis surface. For example, a bioactive agent material can be posited on the surface of the valve prosthesis and a porous coating layer can be posited over the bioactive agent material. The bioactive agent material can diffuse through the porous coating layer. Multiple porous coating layers and or pore size can be used to control the rate of diffusion of the bioactive agent material. The coating layer can also be nonporous wherein the rate of diffusion of the bioactive agent material through the coating layer is controlled by the rate of dissolution of the bioactive agent material in the coating layer.

The bioactive agent material can also be dispersed throughout the coating layer, by for example, blending the bioactive agent with the polymer solution that forms the coating layer. If the coating layer is biostable, the bioactive agent can diffuse through the coating layer. If the coating layer is biodegradable, the bioactive agent is released upon erosion of the biodegradable coating layer.

Bioactive agents may be bonded to the coating layer directly via a covalent bond or via a linker molecule which covalently links the bioactive agent and the coating layer. Alternatively, the bioactive agent may be bound to the coating layer by ionic interactions including cationic polymer coatings with anionic functionality on bioactive agent, or alternatively anionic polymer coatings with cationic functionality on the bioactive agent. Hydrophobic interactions may also be used to bind the bioactive agent to a hydrophobic portion of the coating layer. The bioactive agent may be modified to include a hydrophobic moiety such as a carbon based moiety, silicon-carbon based moiety or other such hydrophobic moiety. Alternatively, the hydrogen bonding interactions may be used to bind the bioactive agent to the coating layer.

The bioactive agent can optionally be applied to or incorporated in any suitable portion of the medical device. The bioactive agent can be applied to or incorporated in an implantable device, a polymer coating applied to the implantable device, a material attached to the implantable frame or a material forming at least a portion of an implantable material. The bioactive agent can be incorporated within the material forming the medical device, or within pores formed in the surface of the medical device. The implantable medical device can optionally comprise a coating layer containing the bioactive agent, or combinations of multiple coating layers configured to promote a desirable rate of elution of the bioactive from the medical device upon implantation within the body.

A coating layer comprising a bioactive agent can comprise a bioactive agent and a biostable polymer, a biodegradable polymer or any combination thereof. In one embodiment, the bioactive agent is blended with a biostable polymer to deposit the bioactive agent within the porous channels within the biostable polymer that permit elution of the bioactive agent from the medical device upon implantation. Alternatively, a blend of the bioactive and the bioabsorbable polymer can be incorporated within a biostable polymer matrix to permit dissolution of the bioabsorbable polymer through channels or pores in the biostable polymer matrix upon implantation in the body, accompanied by elution of the bioactive agent.

Multiple coating layers can be configured to provide a medical device with a desirable bioactive agent elution rate upon implantation. The implantable medical device can comprise a diffusion layer positioned between a portion of the medical device that comprises a bioactive agent and the portion of the medical device contacting the body upon implantation. For example, the diffusion layer can be a porous layer positioned on top of a coating layer that comprises a bioactive agent. The diffusion layer can also be a porous layer positioned on top of a bioactive agent coated on or incorporated within a portion of the implantable medical device.

A porous diffusion layer is preferably configured to permit diffusion of the bioactive agent from the medical device upon implantation within the body at a desirable elution rate. Prior to implantation in the body, the diffusion layer can be substantially free of the bioactive agent. Alternatively, the diffusion layer can comprise a bioactive agent within pores in the diffusion layer. Optionally, the diffusion layer can comprise a mixture of a biodegradable polymer and a bioactive positioned within pores of a biostable polymer of a diffusion layer. In another embodiment, the porous diffusion layer can comprise a mixture of a biodegradable polymer and a biostable polymer, configured to permit absorption of the biodegradable polymer upon implantation of the medical device to form one or more channels in the biostable polymer to permit an underlying bioactive agent to diffuse through the pores formed in the biostable polymer.

In one aspect of the invention, the bioactive agent is an antithrombogenic bioactive agent. Valve prostheses comprising an antithrombogenic bioactive agent are particularly preferred for implantation in areas of the body that contact blood. An antithrombogenic bioactive agent is any therapeutic agent that inhibits or prevents thrombus formation within a body vessel. The valve prosthesis can comprise any suitable antithrombogenic bioactive agent. Types of antithrombotic bioactive agents include anticoagulants, antiplatelets, and fibrinolytics. Anticoagulants are bioactive agents which act on any of the factors, cofactors, activated factors, or activated cofactors in the biochemical cascade and inhibit the synthesis of fibrin. Antiplatelet bioactive agents inhibit the adhesion, activation, and aggregation of platelets, which are key components of thrombi and play an important role in thrombosis. Fibrinolytic bioactive agents enhance the fibrinolytic cascade or otherwise aid is dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Further examples of antithrombotic bioactive agents include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other bioactive agents such as endothelial progenitor cells or endothelial cells.

Other examples of bioactive coating compounds include antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat. Still other bioactive agents that can be incorporated in or coated on a frame include a PPAR□-agonist, a PPAR □agonist and RXR agonists, as disclosed in published U.S. Patent Application US2004/0073297 to Rohde et al., published on Apr. 15, 2004 and incorporated in its entirety herein by reference.

Other examples of bioactive coating compounds include antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as (GP) II_{b}/IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), tacrolimus, everolimus, azathioprine, mycophenolate mofetil); angiogenic agents: vascular endothelial growth factor (VEGF), fibroblast growth factor (FGF); angiotensin receptor blockers; nitric oxide and nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; endothelial progenitor cells (EPC); angiopeptin; pimecrolimus; angiopeptin; HMG co-enzyme reductase inhibitors (statins); metalloproteinase inhibitors (batimastat); protease inhibitors; antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat. Still other bioactive agents that can be incorporated in or coated on a frame include a PPAR α-agonist, a PPAR δ agonist and RXR agonists, as disclosed in published U.S. Publication Number 2004/0073297A1, published Apr. 15, 2004 and incorporated in its entirety herein by reference.

Device Delivery and Methods of Treatment

The valve prosthesis as described herein can be delivered to any suitable body vessel, including a vein, artery, biliary duct, ureteral vessel, body passage or portion of the alimentary canal. Methods for delivering a medical device as described herein to any suitable body vessel are also provided, such as a vein, artery, biliary duct, ureteral vessel, body passage or portion of the alimentary canal. While many preferred embodiments discussed herein discuss implantation of a medical device in a vein, other embodiments provide for implantation within other body vessels. In another matter of terminology there are many types of body canals, blood vessels, ducts, tubes and other body passages, and the term “vessel” is meant to include all such passages.

In some embodiments, valve prostheses of the present invention having a compressed delivery configuration with a very low profile, small collapsed diameter and great flexibility, may be able to navigate small or tortuous paths through a variety of body vessels. A low-profile valve prosthesis may also be useful in coronary arteries, carotid arteries, vascular aneurysms, and peripheral arteries and veins (e.g., renal, iliac, femoral, popliteal, sublavian, aorta, intercranial, etc.). Other nonvascular applications include gastrointestinal, duodenum, biliary ducts, esophagus, urethra, reproductive tracts, trachea, and respiratory (e.g., bronchial) ducts. These applications may optionally include a sheath covering the valve prosthesis. In one aspect, the valve prostheses described herein are implanted from a portion of a catheter inserted in a body vessel.

Still other embodiments provide methods of treating a subject, which can be animal or human, comprising the step of implanting one or more valve prostheses as described herein. In some embodiments, methods of treating may also include the step of delivering a valve prosthesis to a point of treatment in a body vessel, or deploying a valve prosthesis at the point of treatment. Methods for treating certain conditions are also provided, such as venous valve insufficiency, varicose veins, esophageal reflux, restenosis or atherosclerosis. In some embodiments, the invention relates to methods of treating venous valve-related conditions.

“venous valve-related condition” is any condition presenting symptoms that can be diagnostically associated with improper function of one or more venous valves. In mammalian veins, venous valves are positioned along the length of the vessel in the form of leaflets disposed annularly along the inside wall of the vein which open to permit blood flow toward the heart and close to prevent back flow. Two examples of venous valve-related conditions are chronic venous insufficiency and varicose veins.

In the condition of venous valve insufficiency, the valve leaflets do not function properly. For example, the vein can be too large in relation to the leaflets so that the leaflets cannot come into adequate contact to prevent backflow (primary venous valve insufficiency), or as a result of clotting within the vein that thickens the leaflets (secondary venous valve insufficiency). Incompetent venous valves can result in symptoms such as swelling and varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur, for example, in the superficial venous system, such as the saphenous veins in the leg, or in the deep venous system, such as the femoral and popliteal veins extending along the back of the knee to the groin.

The varicose vein condition consists of dilatation and tortuosity of the superficial veins of the lower limb and resulting cosmetic impairment, pain and ulceration. Primary varicose veins are the result of primary incompetence of the venous valves of the superficial venous system. Secondary varicose veins occur as the result of deep venous hypertension which has damaged the valves of the perforating veins, as well as the deep venous valves. The initial defect in primary varicose veins often involves localized incompetence of a venous valve thus allowing reflux of blood from the deep venous system to the superficial venous system. This incompetence is traditionally thought to arise at the saphenofemoral junction but may also start at the perforators. Thus, gross saphenofemoral valvular dysfunction may be present in even mild varicose veins with competent distal veins. Even in the presence of incompetent perforation, occlusion of the saphenofemoral junction usually normalizes venous pressure.

The initial defect in secondary varicose veins is often incompetence of a venous valve secondary to hypertension in the deep venous system. Since this increased pressure is manifested in the deep and perforating veins, correction of one site of incompetence could clearly be insufficient as other sites of incompetence will be prone to develop. However, repair of the deep vein valves would correct the deep venous hypertension and could potentially correct the secondary valve failure. Apart from the initial defect, the pathophysiology is similar to that of varicose veins.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously.

While the invention has been illustrated and described in detail in the drawings and foregoing description, the same is to be considered as illustrative and not restrictive in character, it being understood that only exemplary embodiments have been shown and described and do not limit the scope of the invention in any manner. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

What is claimed is:

1. A radially expandable artificial valve prosthesis for regulating fluid flow through a body vessel, comprising:
a ring frame, wherein the ring frame is radially expandable to form an expanded ring frame having an expanded ring frame width;
a first valve leaflet, wherein a first portion of a perimeter of the valve leaflet is attached to the ring frame to form a valve pocket having a valve pocket height of less than the expanded ring frame width, and
a support structure attached to the ring frame, wherein the support structure positions the ring frame within the bodily vessel,
wherein the first valve leaflet allows fluid flow in a first, antegrade, direction and restricts flow in a second, retrograde direction.

2. The radially expandable artificial valve prosthesis of claim 1, wherein the first valve leaflet is deformable from a first position allowing fluid flow in a first, antegrade, direction to a second position restricting fluid flow in a second, retrograde, direction.

3. The radially expandable artificial valve prosthesis of claim 2, wherein the valve leaflet is positioned so as to create retrograde flow vortices sufficient to reduce stagnation of fluid in the valve pocket when the valve prosthesis is configured to restrict fluid flow in the retrograde direction.

4. The radially expandable artificial valve prosthesis of claim 1, wherein the first portion of a perimeter of the valve leaflet is attached to the ring frame by a method selected from the group consisting of suturing, tissue welding and adhesive bonding.

5. The radially expandable artificial valve prosthesis of claim 1, wherein the ring frame comprises a polymeric material.

6. The radially expandable artificial valve prosthesis of claim 1, wherein the ring frame comprises material selected from a group consisting of stainless steel, nickel, silver, platinum, gold, titanium, tantalum, iridium, tungsten, a self-expanding nickel titanium alloy, and inconel.

7. The radially expandable artificial valve prosthesis of claim 6, wherein the ring frame comprises a self-expanding nickel titanium alloy.

8. The radially expandable artificial valve prosthesis of claim 1, wherein the valve pocket height is less than 40 percent of the ring frame width.

9. The radially expandable artificial valve prosthesis of claim 8, wherein the height of the valve pocket height is less than 30 percent of the ring frame width.

10. The radially expandable artificial valve prosthesis of claim 9, wherein the valve pocket height is less than 15 percent of the ring frame width.

11. The radially expandable artificial valve prosthesis of claim 10, wherein the valve pocket height is less than 10 percent of the ring frame width.

12. The radially expandable artificial valve prosthesis of claim 1, wherein the expanded ring frame forms a substantially planar structure.

13. The radially expandable artificial valve prosthesis of claim 1, wherein a second portion of the perimeter of the valve leaflet is not attached to the ring frame.

14. The radially expandable artificial valve prosthesis of claim 13, wherein the second portion of the perimeter of the valve leaflet is extendable beyond the perimeter of the expanded ring frame.

15. The radially expandable artificial valve prosthesis of claim 14, the perimeter of the valve leaflet further comprising a third portion, wherein the third portion is not attached to the ring frame and wherein the third portion is adapted to allow limited retrograde fluid flow.

16. The radially expandable artificial valve prosthesis of claim 1, wherein at least a portion of the support structure is adapted to expand upon deployment to create an artificial sinus in the bodily passage adjacent to the artificial valve prosthesis and wherein the ring frame is positioned within the artificial sinus.

17. The radially expandable artificial valve prosthesis of claim 1, wherein the ring frame comprises a first ring frame portion and a second ring frame portion forming a continuous ring frame, wherein a first portion of the perimeter of the first valve leaflet is attached to the first ring frame portion and a first portion of a perimeter of a second valve leaflet is attached to the second ring frame portion, and wherein a second portion of the perimeter of the first valve leaflet and a second portion of the perimeter of the second valve leaflet define a lumen allowing antegrade fluid flow in the body vessel.

18. The radially expandable artificial valve prosthesis of claim 1, where the valve leaflet comprises a material selected from the group consisting of a synthetic biocompatible polymer, cellulose acetate, cellulose nitrate, silicone, polyethylene, teraphthalate, polyurethane, polyamide, polyester, polyorthoester, poly anhydride, polyether sulfone, polycarbonate, polypropylene, high molecular weight polyethylene, a fluoroplastic material, polytetrafluoroethylene, or mixtures or copolymers thereof; polylactic acid, polyglycolic acid or copolymers thereof, a polyanhydride, polycaprolactone, polyhydroxy-butyrate valerate, polyhydroxyalkanoate, a polyetherurethane urea, naturally derived or synthetic collagenous material, an extracellular matrix material, submucosa, small intestinal submucosa, stomach submucosa, urinary bladder submucosa, uterine submucosa, renal capsule membrane, dura mater, pericardium, serosa, peritoneum or basement membrane materials, and liver basement membrane.

19. The radially expandable artificial valve prosthesis of claim 1, where the valve leaflet comprises a bioremodelable material.

20. The radially expandable artificial valve prosthesis of claim 1, wherein the valve leaflet comprises small intestinal submucosa.
